(19) United States
(12) Patent Application Publication
(10) Pub. No.: US 2005/0013806 A1
(43) Pub. Date: Jan. 20, 2005

(54) Use of contact lens for corneal cell transplant

(76) Inventors: Min S. Chang, Nashville, TN (US); Rick Haselton, Nashville, TN (US)

Correspondence Address: FULBRIGHT & JAWORSKI L.L.P., 600 CONGRESS AVE., SUITE 2400, AUSTIN, TX, 78701, US

(21) Appl. No.: 10/818,443

(22) Filed: Apr. 5, 2004

Related U.S. Application Data
(60) Provisional application No. 60/460,531, filed on Apr. 4, 2003.

(51) Int. Cl. A61K 48/00; C12N 5/08

(52) U.S. Cl. 424/93.21; 424/93.7; 435/368

(57) ABSTRACT

The present invention addresses the need for improved methods and apparatuses of transplanting cells to injured or diseased cornea. The methods disclosed provide for culture of cells on the concave surface of a contact lens, followed by application of the contact lens to the eye, thereby permitting repopulation of the corneal region by the cells. A particular use for the present invention involves the transfer of genetically altered cells.

This patent application claims priority to, and incorporates by reference, U.S. provisional patent application Ser. No. 60/460,531 filed on Apr. 4, 2003, entitled, “Use of Contact Lens for Corneal Cell Transplant.”

The government owns rights in the present invention pursuant to grant number K08 EY13592-02.

### BACKGROUND OF THE INVENTION

1. Field of the Invention

The present invention relates generally to the fields of ocular medicine and gene therapy. More particularly, it concerns the corneal grafting using contact lens or similar biocompatible matrices.

2. Description of Related Art

The cornea, along with the lens, comprise a critical focusing function for the human eye. The cornea is a thin shell only about 520 micrometers thick. Corneal epithelial cells, which cover the anterior surface of the cornea, account for about 50 micrometers of this thickness. Underlying the epithelium is a layer called Bowman's membrane which is about 20 micrometers thick. The remainder includes the stroma, which is made up of collagen fibers, and a posterior surface comprising non-reproductive endothelial cells.

Current methods of corneal adult stem cell transplantation using the amniotic membrane involve a number of technically difficult steps. First, they require the ex vivo expansion of harvested adult corneal stem cells. These cells are then cultured onto a human amniotic membrane that requires the use of a suitable human amniotic membrane. After corneal stem cells are confluent on the amniotic membrane, the membrane is sutured into place on the damaged corneal surface. The amniotic membrane is very delicate and its placement is technically complicated, requiring microsurgical instruments and specialized suture material. If the suture fails, or the membrane is not tightly adherent, the stem cell transplantation is not successful.

Another limiting feature of this technique is that the amniotic membrane be placed with cell surface up, i.e., with the stem cells separated from the corneal surface by the amniotic membrane. In order for stem cells to take up residence on the corneal, either the amniotic membrane must first degrade, or the stem cells migrate around the edge amniotic membrane onto the ocular surface. Thus, there remains a need in the art to circumvent the use of cumbersome and technically limiting protocols for the treatment of ocular conditions.

### SUMMARY OF THE INVENTION

Thus, in accordance with the present invention, there is provided a method of transplanting cells to the cornea of a subject comprising (a) culturing said cells on the concave surface of a contact lens; and (b) placing said contact lens onto said subject's cornea. The cells may comprise corneal stem cells, such as adult stem cells, and in particular, human cells. The cells also may be genetically-altered cells, for example, with an exogenous gene for a growth factor, e.g., epidermal growth factor. Examples of genetically altered cells include ocular fibroblasts, corneal adult cells, corneal stem cells, or skin fibroblasts. The altered cells may be transformed with an exogenous gene by liposome-mediated transfer, gene gun or viral vector transformation.

Culturing may comprise culturing in serum-free media or on silicon polymers, such as poly-dimethyl silicon. Cells may be aliquoted in media on to the surface of said contact lens, or the contact lens may be immersed in media comprising suspended cells. The contact lens may be a soft lens, a gas permeable lens, a rigid gas permeable (RGP) lens, or an oxygen permeable lens. The contact lens may be pretreated with fibronectin or an adhesive molecule. In particular, the subject may be a human being.

In another embodiment, there is provided a method of providing ocular gene therapy to a patient with an ocular condition comprising the steps of (a) culturing cells on the concave surface of a contact lens; and (b) placing said contact lens onto the patient's cornea. The cells may comprise corneal stem cells, such as adult stem cells, and in particular human cells. The cells may be genetically-altered cells. The ocular condition may comprise ocular inflammation, ocular infection, ocular injury or ocular deficiency (thermal or chemical injury; auto-immune disease, bacterial, viral or fungal infection).

The contact lens may be a soft lens, a gas permeable lens, a rigid gas permeable (RGP) lens, or an oxygen permeable lens. The contact lens may be pretreated with fibronectin or an adhesive molecule. In particular, the subject may be a human being. The method may comprise a second therapy, such as a drug therapy or a second gene therapy. The second therapy may be administered before said ocular gene therapy, along with said ocular gene therapy, or after said ocular gene therapy.

In another embodiment, there is provided a method of transplanting cells to the cornea of a subject comprising (a) culturing said cells in a biocompatible matrix; and (b) placing said matrix onto said subject's cornea. The biocompatible matrix may be a gelatin matrix. The cells may comprise corneal stem cells, such as adult stem cells, and in particular human cells. The cells may be genetically-altered cells. The biocompatible matrix may be covered with a contact lens. The subject may be suffering from an ocular condition such as ocular inflammation, ocular infection, ocular injury or ocular deficiency. The subject may be a human.

In still yet another embodiment, there is provided an intraocular device comprising (a) a lens comprised of a biocompatible material and (b) corneal stem cells. The biocompatible material may be selected from a group comprising of silicone polymer, polydimethylsiloxane, hefilcon, hioxifilicon, methafilcon, and lidofilcon. The corneal stem cells may comprise adult stems cells, in particular human cells. The cells may be genetically-altered cells. In yet another embodiment, there is provided a kit comprising, in a suitable container vessel, (a) a contact lens comprising a concave surface and a convex surface, wherein cells are adherent to the concave surface of said contact lens, and (b) a physiologic medium.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Concave lens molded from Sylgard.

FIG. 2—Human corneal epithelial cells cultured on the concave surface of PDMS lens material.

FIG. 3—Epithelial cells grown on Sylgard and tissue culture plastic exhibit similar growth characteristics.

FIG. 4—Migration of epithelial cells from Sylgard onto tissue culture plastic. The Sylgard appears in the upper left part of the figure and the general direction of the migration is indicated by the curved arrow labeled “migration.” Several epithelial cells are visible on the surface of the plastic and appear to be well-adhered and migrating into regions of lower cell density.

FIG. 5A—Side view of a concave lens with an irregular surface that includes a plurality of valves.

FIG. 5B—Top view of a lens with a plurality of valves.

### DESCRIPTION OF ILLUSTRATIVE EMBODIMENTS

The cornea is an essential part of the human vision system, accounting for about 80% of the focusing ability. Unfortunately, it is subject to a wide variety of genetic, infectious and mechanical damage. For example, though trauma to the cornea often heals rapidly, scarring can cloud the cornea or cause it to lose its proper shape and/or size. Infections of the eye by bacteria, viruses or fungi can cause ulcers on the corneal surface, and even penetrate into the stroma, which leads to inflammation and scaring. Genetic or acquired disorders include primary dysfunctions such as aniridia and congenital erythrokeratoderma. Autoimmune diseases account for additional damage (e.g., Stevens Johnson syndrome). It is well recognized that simply replacing the cornea through corneal transplantation in these circumstances is almost uniformly unsuccessful. Rather, it is necessary for ocular surface to be re-established with normal, corneally-derived epithelium.

### I. The Present Invention

The human cornea is a specialized non-keratinized stratified epithelial tissue, which function as the main refracting element of the eye. In order to maintain its optical properties, the cornea must be transparent and with a regular surface. However, as with other epithelial surfaces in the body, there is constant shedding of epithelial cells; the corneal epithelium turns over every 7 to 10 days. These cells are replenished by stem cells, which reside in the limbus, the junction of the clear cornea and the sclera. If limbal stem cells become deficient, the health of the eye is compromised and is prone to persistent corneal epithelial defects, corneal vascularization, and opacification. Limbal stem cell deficiency can be cause by various conditions including chemical injuries such as alkali burns, trauma, chronic topical medication, chronic corneal exposure, and chronic inflammation.

Current treatments for stem cell deficiency include stem cell allograft transplantation (harvested cadaver tissue) and more recently, using cell-culturing techniques, autograft from the contralateral healthy eye. Autograft transplantation involves harvesting and the ex-vivo expansion of stem cells from the patient and returning the cells back to the patient in the diseased eye. Both methods for treatment of limbal stem cell deficiency are technically difficult requiring specialized microsurgical skills and instruments.

The present invention overcomes numerous drawbacks in current corneal cell transplantation procedures. The present invention uses normal or genetically altered cells that are cultured on the inner surface of a soft contact lens. After the cell culture has been established on the contact lens, the contact lens is placed onto the recipient's cornea. While the contact is resident on the cornea, the stem cells can migrate from the contact lens to take up a residence on the corneal surface, while the contact lens serves to protect the migrated cells while they are establishing residence on the corneal surface. In other embodiment, the cells may preferentially remain on the contact lens and produce biologically active substances which are therapeutic to the cornea. It is contemplated that a variety of cell types, some of which have been genetically altered, will be suitable for this purpose.

Thus, the present method has the following advantages over the other corneal stem cell transplantation techniques: (i) ease of placement of the contact lens, which does not require suturing or specialized instruments, (ii) cells are in direct contact with the cornea, facilitating seeding of stem cells, (iii) reduction of surgical complications such as infection, bleeding, and corneal perforation, and (iv) applicability for use in ocular gene therapy.

### II. Transplantation of Cells to Cornea

The absence or malfunction of corneal stem cells is characterized by the loss of proliferative capacity of corneal epithelium, resulting in surfacing of the cornea with “transdifferentiated” conjunctivally-derived epithelium or, in the worst case, failure to resurface at all in the presence of a persistent epithelial defect, with corneal neovascularization and scarring. The present invention contemplates a method of transplanting cells such as corneal stem cells to the eye of a subject having an ocular condition by culturing cells on a contact lens and placing the contact lens onto the subject's eye. An ocular condition is any abnormal condition of the eye such as an injury, an inflammation, a wound, a defect as will be described later in the specification.

A. Types of cells

The present invention contemplates the use of a wide variety of cells. In a particular embodiment, adult corneal stem cells from humans may be utilized. However, the present inventors envision the use of other cells including fibroblasts and genetically altered cells.

1. Corneal Stem Cells

Corneal epithelium is subject to constant trauma and shedding of the surface epithelium, and replenishment is from epithelial cells beneath and peripheral to the central desquamating epithelium. The origin of the corneal epithelium appears to reside in the crypts of Vogt, where a population of “immortal” stem cells resides, possessing enormous potential for clonogenic cell division. These cells, like all stem cells, have inherent properties which enable them to accomplish error-free replication which avoids development of abnormal differentiation and cellular dysfunction; a low mitotic rate and asymmetrical DNA segregation are essential in this error-free proliferation of these relatively primitive, poorly differentiated cells. The corneal epithelium is maintained by cellular proliferation of these cells which then migrate further onto the central cornea and become terminally differentiated.

A common source for human corneal cells are human corneas found not to be suitable for transplantation. The may be obtained from local eye banks. Excess conjunctival and scleral tissues are removed, leaving approximately 1-2 mm of sclera. A limbal strip is obtained using a scissor cutting 1-2 mm into the clear cornea. The limbal ribbon is further cut into square pieces (about 2-4 mm²). The limbal squares are place cell side down onto a petri dish coated with fibronectin and covered with a commercially available culture medium (Epi-life with HCGS supplement, Cascade Biologic, Portland, Oreg.). The explanted limbal tissues are left undisturbed in a culture incubator for one week after which the medium is changed and the explanted limbal tissues are removed from the petri dish. The corneal epithelial cells are allowed to proliferate up to between 50% to 80% confluency. The cells are treated with trypsin and are further subcultured or stored in liquid nitrogen for future use.

2. Fibroblasts

To obtain fibroblasts, limbal explants from human corneal cell isolation are placed onto a sterile petri dish and covered with the culture medium. These secondary explants are left undisturbed for 1 week prior to first medium change. Afterwards, the medium is changed every 3-4 days. Typically, after 2 weeks the fibroblasts will grow from the limbal tissue and rapidly out number the epithelial cells. Use of a medium containing growth factors can enhance fibroblast cell division. The fibroblast cells are stored in liquid nitrogen for future use.

B. Genetically Altered Cells

In another embodiment, genetically altered cells that express protein of value may also be used in the present method. Such cells may comprise growth factors such as epidermal growth factors, a patient's own cells that are harvested and then cultured, fibroblast from the eye, corneal stem cells, or permanently altered corneal stem cells. Such cells are altered genetically with the corrected gene product.

The ideal gene delivery vector should be capable of efficiently delivering one or more genes of the size needed for clinical application. The vector should be very specific, unrecognized by the immune system, stable, highly reproducible, and be purified in large quantities at high concentrations. Once the vector is inserted into the patient it should not induce an allergic reaction-like inflammation, it should be safe not only for the patient, but also for the environment. Finally, a vector should also be able to express the gene for as long as it is required, ideally the life of the patient.

1. Therapeutic Genes

The present invention contemplates use of a variety of growth factors as therapeutic genes that can be introduced into the corneal cells. Such factors include EGF, substance P, insulin-like gf-1, hepatocyte growth factor, and keratinocyte growth factor.

2. Methods of Making Altered Cells

Methods for delivering a gene into cells are well known to a person of ordinary skill in the art such as chemical methods (Graham et al., 1973; Zatloukal et al., 1992); physical methods such as microinjection (Capecchi, 1980), electroporation (Folger et al., 1982; Fromm et al., 1985; U.S. Pat. No. 5,384,253) and the gene gun (Johnston et al., 1994; Fynan et al., 1993); and viral vectors (Clapp, 1993; Lu et al., 1993; Eglitis et al., 1988a; 1988b).

a. Electroporation

In certain preferred embodiments of the present invention, the gene construct is introduced into the cell to be genetically altered via electroporation. Electroporation involves the exposure of a suspension of cells and DNA to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

It is contemplated that electroporation conditions for the cells that are contemplated to be used in the present invention may be optimized. One may particularly wish to optimize such parameters as the voltage, the capacitance, the time and the electroporation media composition. The execution of other routine adjustments will be known to those of skill in the art.

b. Particle Bombardment

Another embodiment of the invention for transferring a naked DNA construct into cells involves particle bombardment. This method depends on the ability to accelerate DNA-coated microprojectiles to a high velocity allowing them to pierce cell membranes and enter cells without killing them (Klein et al., 1987). The microprojectiles used have consisted of biologically inert substances such as tungsten, platinum or gold beads.

It is contemplated that in some instances DNA precipitation onto metal particles would not be necessary for DNA delivery to a recipient cell using particle bombardment. It is contemplated that particles may contain DNA rather than be coated with DNA. Hence it is proposed that DNA-coated particles may increase the level of DNA delivery via particle bombardment but are not, in and of themselves, necessary.

Several devices for accelerating small particles have been developed. One such device relies on a high voltage discharge to generate an electrical current, which in turn provides the motive force (Yang et al., 1990). Another method involves the use of a Biolistic Particle Delivery System, which can be used to propel particles coated with DNA through a screen, such as stainless steel or Nytex screen, onto a filter surface covered with cells in suspension. The screen disperses the particles so that they are not delivered to the recipient cells in large aggregates. It is believed that a screen intervening between the projectile apparatus and the cells to be bombarded reduces the size of projectile aggregates and may contribute to a higher frequency of transformation by reducing the damage inflicted on the recipient cells by projectiles that are too large.

For the bombardment, cells in suspension are preferably concentrated on filters, or alternatively on solid culture medium. The cells to be bombarded are positioned at an appropriate distance below the macroprojectile stopping plate. If desired, one or more screens are also positioned between the acceleration device and the cells to be bombarded.

In bombardment transformation, one may optimize the prebombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity or either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of primordial germ cells.

Accordingly, it is contemplated that one may wish to adjust various bombardment parameters in small scale studies to fully optimize the conditions. One may particularly wish to adjust physical parameters such as gap distance, flight distance, tissue distance and helium pressure. One also may optimize the trauma reduction factors by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art.

c. Calcium Phosphate Co-Precipitation or DEAE-Dextran Treatment

In other embodiments of the present invention, the transgenic construct is introduced to the cells using calcium phosphate co-precipitation. Mouse primordial germ cells have been transfected with the SV40 large T antigen, with excellent results (Watanabe et al., 1997). Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and erythroleukemia cells (Gopal, 1985).

d. Direct Microinjection or Sonication Loading

Further embodiments of the present invention include the introduction of the nucleic acid construct by direct microinjection or sonication loading. Direct microinjection has been used to introduce nucleic acid constructs into Xenopus ocytes (Harland and Weintraub, 1985), and LTK-fibroblasts have been transfected with the thymidine kinase gene by sonication loading (Fechheimer et al., 1987).

e. Lipid-Mediated Transformation

In a further embodiment of the invention, the gene construct may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in lipid vehicle stability in the presence and absence of serum proteins. The interaction between lipid vehicles and serum proteins has a dramatic impact on the stability characteristics of lipid vehicles (Yang and Huang, 1997). Cationic lipids attract and bind negatively charged serum proteins. Lipid vehicles associated with serum proteins are either dissolved or taken up by macrophages leading to their removal from circulation. Current in vivo lipid delivery methods use subcutaneous, intradermal, intratumoral, or intracranial injection to avoid the toxicity and stability problems associated with cationic lipids in the circulation. The interaction of lipid vehicles and plasma proteins is responsible for the disparity between the efficiency of in vitro (Felgner et al., 1987) and in vivo gene transfer (Zhu et al., 1993; Philip et al., 1993; Solodin et al., 1995; Thierry et al., 1995; Tsukamoto et al., 1995; Aksentijevich et al, 1996).

Recent advances in lipid formulations have improved the efficiency of gene transfer in vivo (Smyth-Templeton et al., 2002; WO 98/07408). A novel lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome”. This formulation is reported to “sandwich” DNA between an invaginated bi-layer or “vase” structure. Beneficial characteristics of these lipid structures include a positive colloidal stabilization by cholesterol, two dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Lipid encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating angiogenesis-related diseases.

f. Viral Transformation

Adenoviral Infection. One method for delivery of the recombinant DNA involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a recombinant gene construct that has been cloned therein.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus et al., 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus et al., 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Retroviral Infection. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

AAV Infection. Adeno-associated virus (AAV) is an attractive vector system for use in the present invention as it has a high frequency of integration and it can infect non-dividing cells, thus making it useful for delivery of genes into mammalian cells in tissue culture (Muzyczka, 1992). AAV has a broad host range for infectivity (Tratschin et al, 1984; Laughlin et al., 1986; Lebkowski et al., 1988; McLaughlin et al., 1988), which means it is applicable for use with the present invention. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Luo et al., 1996; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al, 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

Other Viral Vectors. Other viral vectors may be employed as constructs in the present invention. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990).

A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been suggested that VEE may be an extremely useful vector for immunizations (Caley et al., 1997).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. (1991) recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In still further embodiments of the present invention, the nucleic acid encoding extracellular human MDA-7 to be delivered is housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

For example, to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro Roux et al., 1989).

C. Contact Lens and other Biocompatible Materials

The present invention contemplates the use of a contact lens or other biocompatible material suitable as substrates for culturing the cells. Further, the contact lenses or biocompatible material may also serve as a delivery vehicle to repopulate the cornea by direct transfer. Soft contact lenses are the most commonly prescribed type of contact lenses. Around 90% of all the contact lenses prescribed are soft lenses. Soft contact lenses are made from soft, flexible plastics, known as hydrogels, which contain water. The water allows oxygen to pass through the lens to the eye. This is important, as the cornea (the clear front surface of the eye) obtains its oxygen supply directly from the air. Thus, soft contact lenses provide an ideal substrate for cell culturing and transplantation.

In one embodiment, the soft contact lens will comprise a silicone polymer, such as dimethyl silicon. Polymacon was the original soft contact lens material and is used for daily, disposable, and extended wear. It is a low-water, non-ionic lens material made of a homo-polymer of 2-hydroxyethyl mathacrylate. Following hydration, lenses contain 38% water by weight.

Hefilcon is another popular soft lens material, made of a co-polymer of HEMA (2-hydroxyethyl methacrylate) and NVP (N-vinyl-2-pyrrolidone). It also is a low-water, non-ionic material, containing 45% water by weight and are softer and more flexible than polymacon lenses.

Hioxifilcon is a co-polymer of HEMA (2-hydroxyethyl methacrylate) and GMA (glyceryl monomethacrylate). GMA was one originally investigated for optical applications in the '60s, but not used in contact lens formulations until recently due to difficulties in purification and lack of commercial availability. A non-ionic polymer, Hioxifilcon has improved dimensional stability and improved water balance. Lenses contain 48% water by weight when hydrated.

Methafilcon is used to manufacture daily and extended wear lenses. It is a co-polymer of HEMA (2-hydroxyethyl methacrylate) and MAA (methacrylic acid), and is classified as an ionic, high-water material. Lenses made from this material are noted for their high strength and stability, which makes it the preferred material for toric lenses.

Lidofilcon was developed specifically for extended wear applications. It is a co-polymer of MMA (methyl methacrylate) and NVP (N-vinyl-2-pyrrolidone). Containing 70% water by weight, it is classified as a high-water, non-ionic material. These lenses feel softer and more flexible than other hydrogel lenses.

The present inventors also have grown immortalized human corneal epithelial cells on silicone elastomer. Though not the usual material for soft contact lenses, Bausch and Lomb produces a contact lens call Silsoft™ from this material. Lightman, J M and Marshall, D, 1996, 73(1) 22-27, Optometry and Vision Sciences. One example of a silicone elastomer is PMDS (polydimethylsiloxane) which has been identified by the present inventors as a suitable material for culturing cells. PDMS is highly oxygen permeable, allowing oxygen delivery to cells of the cornea. Further, PDMS exhibits external biocompatibility properties, promoting cell attachment and growth in vitro.

Rigid lenses are made from plastics which are less flexible than those used for soft lenses. While the earliest materials used to make these lenses did not allow any oxygen to pass through the lens, the materials used today are permeable to oxygen, which is why these lenses are most commonly referred to as “rigid gas permeable” or “RGP” lenses. Some of the newer materials allow almost as much oxygen to reach the eye as if no lens was being worn. Nevertheless, rigid lenses can move more easily on the eye as compared to soft lenses which, when sized appropriately to cover the whole eye, and will not move or move only minimally. However, there may be certain applications when additional support or protection is desired, and rigid lenses will be preferred.

In an alternative embodiment, the invention also contemplates the pretreatment of lenses prior to culturing of cells. For example, biomaterials that provide structural support for proliferating cells may prove helpful. In particular, the inventors contemplate the use of fibronectin and other materials that will aid in cell adhesion.

D. Culturing Cells on a Contact Lens and Transferring the Cultured Cells to a Corneal Surface

Cells may be cultured in a contact lens in two general fashions. First is what one can term the “cup” method. This approach makes use of the concave surface of the contact lens as a well in which one can culture the cells. This method has the advantage of wasting very little medium or cells, and would be particularly useful in the context of transplanting autologous cells back into a patient's eye.

Alternatively, cells may be cultured using an “immersion” method, where the entire lens is immersed, concave side up, in media in which cells are suspended. Cells will then settle onto the concave surface of the lens. This method has particular value in scale up procedures where the lenses are mass-produced and then pre-packaged. Such lenses may be placed in a “kit” that comprises receptacles for individual or pairs of lenses, preferably in a liquid-tight container so that the lenses may be immersed in a physiologic media to preserve cellular integrity.

The contact lens with the cultured cells may directly be placed in contact with a recipient's corneal surface. The cultured cells may migrate from the lens and may take residence on surface while the contact lens serve as a protective barrier during the migration.

Similarly, the inventors contemplated using contact lens that may include prongs protruding from the concave surface of the lens, where the prongs may be used for direct delivery of the cells attached to the lens to a corneal surface. For instance, a plurality of pedicles may be made of a silicon polymer micro-fabricated onto a surface of the lens. These posts may project from the lens and may facilitate the transfer of nearby cells attached to the lens. Particularly, the cells may migrate along the posts towards the corneal surface. This structure has the advantage of increasing the number of cells implanted onto the surface as well as reduce the therapy time.

E. Biocompatible Materials

In another embodiment, the present invention contemplates the use of biocompatible materials as supports for corneal cells. In particular, the inventors envision the use of amniotic membranes (AM), obtained from cesarean sections. AM epithelium is removed by chemical processing with 10% ammonium for 15 minutes followed by gentle scraping with surgical blades. This method removes almost all AM epithelium, but leaves basement membrane structures intact. The AM is then placed on a surface (e.g., culture dish or well). Shimazaki et al. (2002); Tsubota et al. (1996); Shimazaki et al. (1997); Tsubota et al. (1999).

Another material suitable for use as a culture substrate is fibrin. Fibrin is a highly manageable and quickly degradable substrate for culture of corneal cells. Rama et al. (2001). Fibrin sealant, used to create the substrate, is made by mixing equal volumes of thrombin and fibrinogen, as described in Pellegrini et al. (1999).

### III. Gene Therapy for Ocular Conditions

There are various conditions related to the eye that may result in ocular pathology such as an ocular wound, an ocular inflammation, ocular infection, ocular injury or an ocular deficiency. An ocular wound may occur due to physical means of injury to the eye such as a blow, or being hit by a sharp object. Ocular inflammation may occur due to side effects of medication or due to an immunological response arising due to various causes, including autoimmunity. An ocular infection may arise due to infection of the eye by a virus, a bacteria, a fungi or a protozoa. An ocular injury may be attributed to a thermal or a chemical cause. An ocular deficiency could arise due to a genetic defect in the body.

Gene therapy is basically the repairing of genes to correct for diseases that result from a loss or change in our genetic material. There are three sequential steps to gene therapy. The first step, which is optional, includes removal of a patient's own cells. This step has an advantage in avoiding immune reaction against the cells on retransplated. Second, one or more genes are inserted into the recipient cells. Third, the genetically-modified cells are transferred back to the patient once the genes have been fixed in their vectors (Gardner et al., 1991).

It is envisioned that ocular gene therapy can be applied in two ways. First, the cells may be engineered to overcome a genetic defect in the host's own cells. This traditional application of gene therapy generally requires knowledge of the genetic defect, although empirical study revealing the benefits of a particular gene in a genetic disease may also provide a therapeutic strategy. The second way gene therapy may be applied is in the provision of therapeutic product. For example, there are proteins that may aid in wound healing or in the treatment of an infection. In some cases, these proteins would be engineered so that they can be secreted by the transplanted cells. A variety of gene transfer techniques have been described above.

To deliver vector two techniques have been used: ex vivo and in vivo. The ex vivo method (outside the living body) uses extracted cells from the patient. First, the normal genes are inserted into vectors, and the target cells with defective genes are removed from the patient. The cells and vectors contacted under conditions permitting uptake and expression, and the genetically engineered cells are transplanted back to the patient.

The in vivo method does not use cells from the patient's body, thus eliminating expensive, and very time consuming laboratory procedures. Rather, other cells, perhaps even those pre-transformed with the therapeutic gene, are provided to the patient. While convenient, such cells may suffer the drawback of eliciting an immune reaction from the host.

As an example of the first kind of therapy, the genetic defect for several anterior stromal corneal dystrophies has been identified as defects BIGH3 gene. These corneal dystrophies include lattice corneal dystrophy, granular corneal dystrophy, Avellino corneal dystrophy and Reis-Buckler corneal dystrophy. With the use of use stem cell transferring technique, a corrected copy of the BIGH3 gene may be inserted into the patient's ex vivo expanded stem cell and subsequently transplanted back the eye and correcting the genetic defect on the ocular surface.

The second kind of gene therapy, referred to as “genetic pharmaceuticals,” uses the contact lens as a bio-reactors. The cells from the patient can be transfected with various genes to treat different ocular condition. Such treatment include angiogenesis inhibitors, such as endostatin, for the treatment of corneal vacularization caused by trauma, chronic inflammation (severe blephritis, chronic drug exposure, ocular cicatricial pemphigoid, trachoma), corneal transplantation, post-surgical trauma, and corneal infection (bacterial keratitis, viral keratitis). Another example is the use of epithelial growth factor for treatment for persistent corneal epithelial defect caused by neurotrophic cornea (from diabetes, congenital causes, or acquired secondary to neuro-surgery or post-herpetic infections) or post-corneal transplantation.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Lens Fabrication

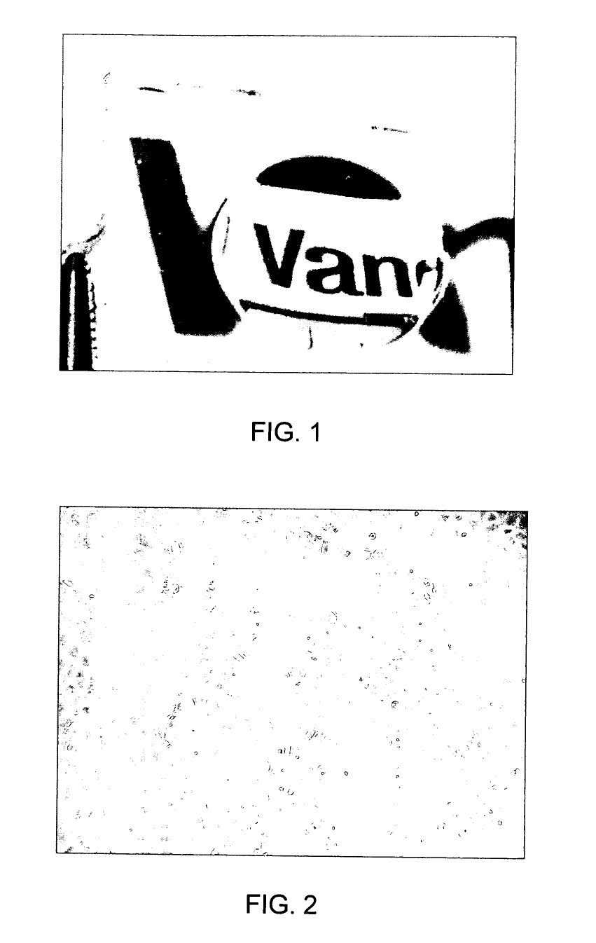

FIG. 1 shows an example of a concave molded lens fabricated from Sylgard 184 (poly (dimethyl siloxane)) (Dow Corning). The lens shown is approximately 2 cm in diameter and less than 1 mm thick and was molded using a standard drop slide following the manufacturer's instruction for the use of this material. A negative of the drop slide indentation was made and the second positive lens made by pouring Sylgard into the first negative mold and removing the material from the negative lens mold before the material had completely polymerized. The concave nature is evident from the distorted text image after the lens was placed onto newsprint.

### Example 2

### Lens Material Supports the Growth of Epithelial Cells

The inventors used a human corneal epithelial cell line immortalized with SV40 to test the cell growth and transfer characteristics. FIG. 2 shows an example of cultured human epithelial cells grown on the inside curvature an untreated surface of Sylgard 184. In these experiments, the molded material was autoclaved and placed into a 100 mm Petri dish with its concave side up. The mold was filled with cells plus media. The media was Gibco's defined keratinocyte media without serum supplementation. Approximately 15,000 cells (or approximately 5,000 cells per cm²) were added to the inside of the mold. Media was changed twice weekly. Cells were cultured for 7-10 days and then photographed. As shown in FIG. 2, the cells appeared confluent but patchy in some areas.

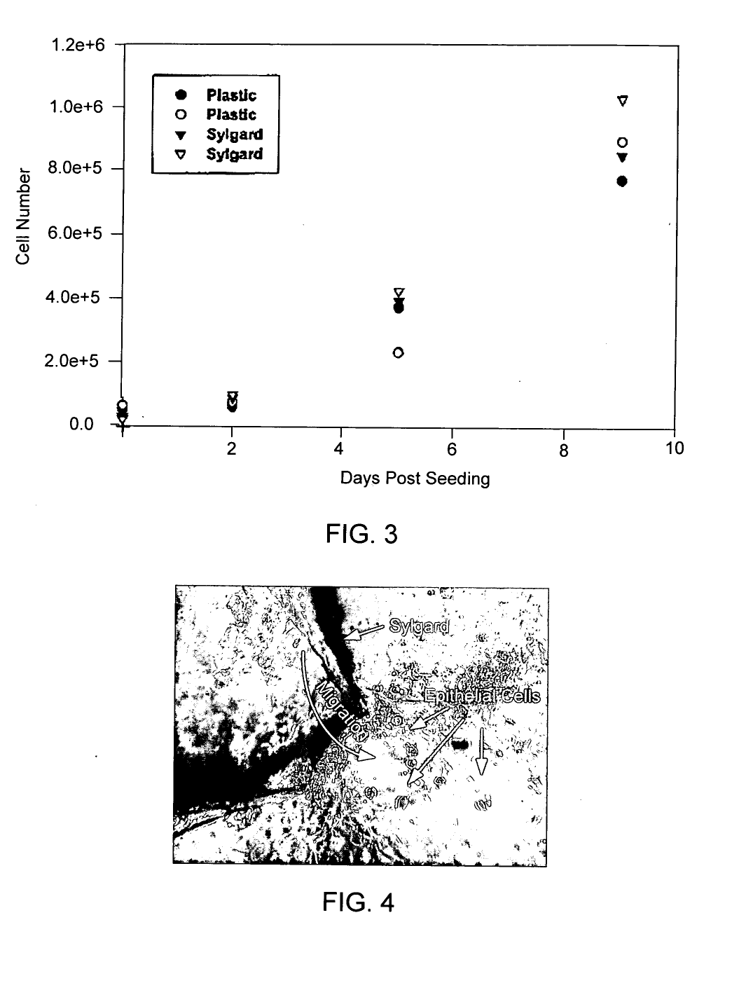

As a further test of the growth characteristics, the inventors compared the growth of Sylgard 184 with standard tissue culture plastic. In two six-well plates, the inventors poured a thin layer of Sylgard 184 and let it polymerize. To these wells and two additional plates of 6-wells, the inventors added 22,500 cells (5,000 cells per cm²) and followed the time course of cell growth. Cells were trypsinized and counted at 2, 5 and 9 days after seeding. As can be seen in FIG. 3, the growth characteristics on plastic and Sylgard were similar.

### Example 3

### Cells Grown on Sylgard Migrate from Sylgard onto Tissue Culture Plastic

In these experiments, the inventors sought to demonstrate that epithelial cells will grow on Sylgard 184 but retain their ability to migrate onto another surface. This will be important in seeking to promote the transfer of cells from the extracorpeal device back onto the corneal surface. In these experiments, Sylgard 184 with epithelial cells grown on their surface were placed into a tissue culture flask cell-side down. Within 2 to 3 days, migration of the cells from the Sylgard onto the plastic was evident. FIG. 4.

### Example 4

### Lens Surface Promotes the Migration of Cells from Lens to Corneal Surface

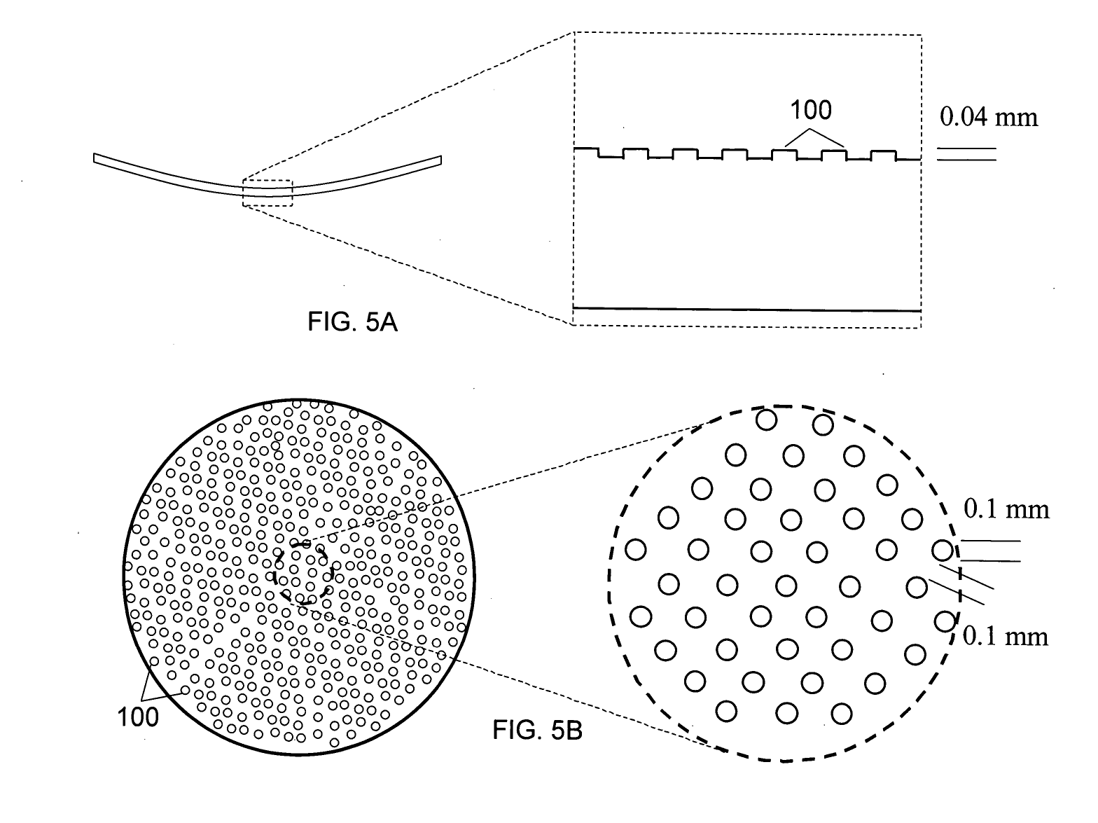

FIG. 5A an example of an irregular-shaped lens surface. The lens shown is approximately 1 cm in diameter and includes a plurality of valves 100 that project from the concave surface of the lens. These valves aid in the migration of the cultured cells from the lens to the corneal surface. Particularly, cells that are attached to the lens surface may utilize a nearby valve to migrate. As such, the lens surface may include a highly-dense valve placement, where each valve is spaced apart from a neighboring valve by approximately 200 microns, center to center, as shown in FIG. 5B.

All of the composition and methods disclosed and claimed herein can be made and executed without undue experimentation in light of the present disclosure. While the compositions and methods of this invention have been described in terms of preferred embodiments, it will be apparent to those of skill in the art that variations may be applied to the composition and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.
U.S. Pat. No. 4,797,368
U.S. Pat. No. 5,139,941
U.S. Pat. No. 5,384,253
Aksentijevich et al., Hum. Gene Ther., 7(9):1111-1122, 1996.
Baichwal and Sugden, In: Gene Transfer, Kucherlapati (ed.), New York, Plenum Press, 117-148, 1986.
Caley et al., J. Virology, 71(4):3031-3038, 1997.
Capecchi, Cell, 22(2 Pt 2):479-488, 1980.
Chang et al., Hepatology, 14:134A, 1991.
Chen and Okayama, Mol. Cell Biol., 7(8):2745-2752, 1987.
Clapp, Clin. Perinatol., 20(1):155-168, 1993.
Clark et al., Hum. Gene Ther., 6(10):1329-1341, 1995.
Coffin, In: Virology, Fields et al., eds., Raven Press, NY, 1437-1500, 1990.
Couch et al., Am. Rev. Resp. Dis., 88:394-403, 1963.
Coupar et al., Gene, 68:1-10, 1988.
Davis et al, Curr. Biol., 6:146-148, 1996.
Eglitis et al., Biotechniques, 6(7):608-614, 1988a.
Eglitis et al., Adv. Exp. Med. Biol., 241:19-27, 1988b.
Fechheimer et al., Proc. Natl. Acad. Sci. USA, 84:8463-8467, 1987.
Felgner et al., Proc. Natl. Acad. Sci. USA, 84(21):7413-7417, 1987.
Flotte et al., Am. J. Respir. Cell Mol. Biol., 7(3):349-356, 1992.
Flotte et al., Gene Ther, 2(1):29-37, 1995.
Flotte et al., Proc. Natl. Acad. Sci. USA, 90(22):10613-10617, 1993.
Folger et al., Mol. Cell. Biol., 2(11):1372-1387, 1982.
Fraley et al., Proc. Natl. Acad. Sci. USA, 76:3348-3352, 1979.
Friedmann, “Progress toward human gene therapy”, Science, 244:1275-1281, 1989.
Fromm et al., Proc. Natl. Acad. Sci. USA, 82(17):5824-5828, 1985.
Fynan et al., Proc. Natl. Acad. Sci. USA, 90(24):11478-11482, 1993.
Gabizon et al., Cancer Res., 50(19):6371-6378, 1990.
Ghosh and Bachhawat, In: Liver Diseases, Targeted Diagnosis and Therapy Using Specific Receptors and Ligands. Wu et al., eds., Marcel Dekker, NY, 87-104, 1991.
Ghosh-Choudhury et al., EMBO J., 6:1733-1739, 1987.
Gomez-Foix et al., J. Biol. Chem., 267:25129-25134, 1992.
Gopal, Mol. Cell Biol., 5:1188-1190, 1985.
Graham and Prevec, In: Methods in Molecular Biology: Gene Transfer and Expression Protocol, Murray (Ed.), Humana Press, Clifton, N.J., 7:109-128, 1991.
Graham and Van Der Eb, Virology, 52:456-467, 1973.
Graham et al, J. Gen. Virl., 36(1):59-74, 1977.
Grunhaus et al., Seminar in Virology, 200(2):535-546, 1992.
Harland and Weintraub, J. Cell Biol., 101:1094-1099, 1985.
Hermonat and Muzycska, Proc. Natl. Acad. Sci. USA, 81:6466-6470, 1984.
Hersdorffer et al., DNA Cell Biol., 9:713-723, 1990.
Herz and Gerard, Proc. Natl. Acad. Sci. USA, 90:2812-2816, 1993.
Horwich et al., Virol., 64:642-650, 1990.
Johnston and Tang, Methods Cell Biol., 43(A):353-365, 1994.
Jones and Shenk, Cell, 13:181-188, 1978.
Kaplitt et al., Nat Genet., 8(2):148-54, 1994.
Karlsson et al., EMBO J., 5:2377-2385, 1986.
Klein et al., Nature, 327:70-73, 1987.
Kotin et al., Proc. Natl. Acad. Sci. USA, 87(6):2211-2215, 1990.
LaFace et al., Virology, 162(2):483-486, 1988.
Laughlin et al., J. Virol., 60(2):515-524, 1986.
Le Gal La Salle et al., Science, 259:988-990, 1993.
Lebkowski et al., Mol. Cell Biol., 8(10):3988-3996, 1988.
Levrero et al., Gene, 101:195-202, 1991.
Lu et al., J. Exp. Med., 178(6):2089-2096, 1993.
Luo et al., Proc. Natl. Acad. Sci. USA, 93:8907-8912, 1996.
Mann et al., Cell, 33:153-159, 1983.
Markowitz et al., J. Virol., 62:1120-1124, 1988.
McCarty et al., J. Virol., 65(6):2936-2945, 1991.
McLaughlin et al., J. Virol., 62(6):1963-1973, 1988.
Muzyczka, Curr. Top Microbiol. Immunol., 158:97-129, 1992.
Nicolas and Rubenstein, In: Vectors: A survey of molecular cloning vectors and their uses, Rodriguez and Denhardt (eds.), Stoneham: Butterworth, 493-513, 1988.
Nicolau and Sene, Biochim. Biophys. Acta, 721:185-190, 1982.
Nicolau et al., Methods Enzymol., 149:157-176, 1987.
Ohi et al., Gene, 89(2):279-282, 1990.
Paskind et al., Virology, 67:242-248, 1975.
Pellegrini et al., Transplantation, 68(6):868-879, 1999.
Philip et al., J. Biol. Chem., 268(22):16087-16090, 1993.
Potter et al., Proc. Natl. Acad. Sci. USA, 81:7161-7165, 1984.
Racher et al., Biotechnology Techniques, 9:169-174, 1995.
Ragot et al., Nature, 361:647-650, 1993.
Rama et al., Transplantation, 72(9):1478-1485, 2001.
Rich et al., Hum. Gene Ther., 4:461-476, 1993.
Ridgeway, In: Vectors: A survey of molecular cloning vectors and their uses. Rodriguez and Denhardt, eds., Stoneham, Butterworth, 467-492, 1988.
Rippe et al., Mol. Cell Biol., 10:689-695, 1990.
Rosenfeld et al, Cell, 68:143-155, 1992.
Rosenfeld et al., Science, 252:431-434, 1991.
Roux et al, Proc. Natl. Acad. Sci. USA, 86:9079-9083, 1989.
Samulski et al., EMBO J., 10:3941-3950, 1991.
Samulski et al., J. Virol., 63:3822-3828, 1989.
Shelling and Smith, Gene Therapy, 1:165-169, 1994.
Shimazaki et al., Ophthalmology, 104(12):2068-2076, 1997.
Shirnazaki et al., Ophthalmology,. 109(7):1285-1290, 2002.
Smyth-Templeton et al., DNA Cell Biol., 21(12):857-867, 2002.
Solodin et al., Biochemistry, 34(41):13537-13544, 1995.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Temin, In: Gene Transfer, Kucherlapati (ed.), NY, Plenum Press, 149-188, 1986.
Thierry et al., Proc. Natl. Acad. Sci. USA, 92(21):9742-9746, 1995.
Top et al., J. Infect. Dis., 124:155-160, 1971.
Tratschin et al., Mol. Cell. Biol., 4:2072-2081, 1984.
Tratschin et al., Mol. Cell Biol., 5:3258-3260, 1985.
Tsubota et al., Am. J. Ophthalmol., 122(1):38-52, 1996.
Tsubota et al., Ophthalmology, 106(10):1984-1989, 1999.
Tsukamoto et al., Nat. Genet., 9(3):243-248, 1995.
Tur-Kaspa et al., Mol. Cell Biol., 6:716-718, 1986.
Walsh et al., J. Clin. Invest., 94:1440-1448, 1994.
Watanabe et al, Exper. Cell Res., 230:76-83, 1997.
Wei et al., Gene Therapy, 1:261-268, 1994.
Wong et al., Gene, 10:87-94, 1980.
Yang and Huang, Gene Therapy, 4 (9):950-960, 1997.
Yang et al., J. Virol., 68:4847-4856, 1994.
Yang et al., Proc Natl. Acad Sci. USA, 87:9568-9572, 1990.
Yoder et al., Blood, 82 (Supp.): 1:347A, 1994.
Zatloukal et al., Ann NY Acad. Sci., 660:136-153, 1992.
Zhou et al., Exp. Hematol., 21:928-933, 1993.
Zhou et al., J. Exp. Med., 179:1867-1875, 1994.
Zhu et al., Science, 261(5118):209-211, 1993.

1. A method of transplanting cells to the cornea of a subject comprising:
(a) culturing said cells on the concave surface of a contact lens; and
(b) placing said contact lens onto said subject's cornea.

2. The method of claim 1, wherein said cells comprise corneal stem cells.

3. The method of claim 2, wherein said corneal stem cells are adult stem cells.

4. The method of claim 3, wherein said adult corneal stem cells are human cells.

5. The method of claim 1, wherein said cells are genetically-altered cells.

6. The method of claim 5, wherein said cells comprise an exogenous gene for a growth factor.

7. The method of claim 6, wherein said growth factor is epidermal growth factor.

8. The method of claim 5, wherein said cells are ocular fibroblasts, corneal adult cells, corneal stem cells, or skin fibroblasts.

9. The method of claim 5, wherein said cells are transformed with an exogenous gene by liposome-mediated transfer, gene gun or viral vector transformation.

10. The method of claim 1, wherein culturing cells comprises culturing in serum-free media.

11. The method of claim 1, wherein culturing cells comprises culturing on silicon polymers.

12. The method of claim 11, wherein the silicon polymers comprise poly-dimethyl silicon.

13. The method of claim 1, wherein cells are aliquoted in media on to the surface of said contact lens.

14. The method of claim 1, wherein said contact lens is immersed in media comprising suspended cells.

15. The method of claim 1, wherein said contact lens is a soft lens.

16. The method of claim 1, wherein said contact lens is gas permeable.

17. The method of claim 16 wherein said contact lens is rigid gas permeable (RGP).

18. The method of claim 16, wherein said contact lens is oxygen permeable.

19. The method of claim 1, wherein said contact lens is pretreated with fibronectin or an adhesive molecule.

20. The method of claim 1, wherein said subject is a human being.

21. A method of providing ocular gene therapy to a patient with an ocular condition comprising the steps of:
(a) culturing cells on the concave surface of a contact lens; and
(b) placing said contact lens onto the patient's cornea.

22. The method of claim 21, wherein said cells comprise corneal stem cells.

23. The method of claim 22, wherein said corneal stem cells are adult stem cells.

24. The method of claim 23, wherein said adult corneal stem cells are human cells.

25. The method of claim 21, wherein said cells are genetically-altered cells.

26. The method of claim 21, wherein said ocular condition comprises ocular inflammation, ocular infection, ocular injury or ocular deficiency.

27. The method of claim 26, wherein said ocular injury is a thermal or chemical injury.

28. The method of claim 26, wherein said ocular inflammation is auto-immune in origin.

29. The method of claim 26, wherein said ocular infection is bacterial, viral or fungal.

30. The method of claim 21 wherein said contact lens is a soft lens.

31. The method of claim 21, wherein said contact lens is gas permeable.

32. The method of claim 31, wherein said contact lens is rigid gas permeable (RGP).

33. The method of claim 31, wherein said contact lens is oxygen permeable.

34. The method of claim 21, wherein said contact lens is pretreated with fibronectin or an adhesive molecule.

35. The method of claim 21, wherein said patient is a human being.

36. The method of claim 21, further comprising a second therapy.

37. The method of claim 36, wherein said second therapy is drug therapy or a second gene therapy.

38. The method of claim 37, wherein said second therapy is administered before said ocular gene therapy.

39. The method of claim 37, wherein said second therapy is administered along with said ocular gene therapy.

40. The method of claim 37, wherein said second therapy is administered after said ocular gene therapy.

41. A kit comprising, in a suitable container vessel, (a) a contact lens comprising a concave surface and a convex surface, wherein cells are adherent to the concave surface of said contact lens, and (b) a physiologic medium.

42. A method of transplanting cells to the cornea of a subject comprising:
(a) culturing said cells in a biocompatible matrix; and
(b) placing said matrix onto said subject's cornea.

43. The method of claim 42, wherein said biocompatible matrix is a gelatin matrix.

44. The method of claim 42, wherein said cells comprise corneal stem cells.

45. The method of claim 44, wherein said corneal stem cells are adult stem cells.

46. The method of claim 45, wherein said adult corneal stem cells are human cells.

47. The method of claim 42, wherein said cells are genetically-altered cells.

48. The method of claim 42, wherein said biocompatible matrix is covered with a contact lens.

49. The method of claim 42, wherein said subject is suffering from an ocular condition such as ocular inflammation, ocular infection, ocular injury or ocular deficiency.

50. The method of claim 42, wherein said subject is a human.

51. An intraocular device, comprising
(a) lens comprised of a biocompatible material; and
(b) corneal stem cells.

52. The intraocular device of claim 51, the biocompatible material being selected from the group consisting of: silicone polymer, polydimethylsiloxane, hefilcon, hioxifilicon, methafilcon, and lidofilcon, 53. The intraocular device of claim 51, wherein said corneal stem cells are adult stem cells.

54. The intraocular device of claim 53, wherein said adult corneal stem cells are human cells.

55. The intraocular device of claim 51, wherein said corneal stems cells are genetically-altered.

56. The intraocular device of claim 51, wherein said lens comprises a plurality of valves.
